(19) United States
(12) Patent Application Publication
(10) Pub. No.: US 2023/0257818 A1
(43) Pub. Date: Aug. 17, 2023

(54) MINIMIZING FETAL FRACTION BIAS IN MATERNAL POLYGENIC RISK SCORE ESTIMATION

(71) Applicant: ILLUMINA, INC., San Diego, CA (US)

(72) Inventors: Michael Mehan, San Diego, CA (US); Sung Kim, San Diego, CA (US); Cosmin Deciu, San Diego, CA (US)

(21) Appl. No.: 18/109,109

(22) Filed: Feb. 13, 2023

Related U.S. Application Data
(60) Provisional application No. 63/310,876, filed on Feb. 16, 2022.

(51) Int. Cl. C12Q 1/6883; G16B 20/20; G16H 50/30

(52) U.S. Cl. CPC C12Q 1/6883; G16B 20/20; G16H 50/30

(57) ABSTRACT

The presently described techniques provide for the use of low-pass sequencing data in the calculation of a polygenic risk score for an individual. As discussed herein, the low-pass sequencing data may be acquired in a context where DNA (e.g., cfDNA) from more than one source is present in the sample and the portion of the DNA attributable to a secondary source may bias the PRS calculation for the primary individual of interest. In one implementation fragment length may be used to derive a function (e.g., a linear function) relating fetal fraction to the respective PRS estimate at each fetal fraction. This function may then be used to calculate the PRS in the absence of a fetal contribution (i.e., at a 0% fetal fraction).

### CROSS REFERENCE TO RELATED APPLICATIONS

This application claims priority to and the benefit of U.S. Provisional Application Ser. No. 63/310,876, entitled “MINIMIZING FETAL FRACTION BIAS IN MATERNAL POLYGENIC RISK SCORE ESTIMATION”, filed Feb. 16, 2022, which is hereby incorporated by reference in its entirety for all purposes.

### BACKGROUND

The present approach relates generally to the use of maternal blood samples, and particularly cell-free DNA (cfDNA) present within such blood samples, to assess the likelihood of various polygenic trait of interest, such as in the mother or father. More particularly, the approach generally relates to limiting or eliminating the confounding effects of non-maternal cfDNA present within the blood sample on the assessment of the polygenic trait of interest, such as a disease or disorder that may be attributed to or effected by multiple loci within the genome.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

There are instances in which a characteristic or condition (e.g., a disease state) of a person may be genetically complex and may have a multitude of genetic components. Such traits, whether corresponding to a disease state or other non-disease condition, may be referred to as polygenic and may be caused by or associated with hundreds to thousands of genetic variants that act in conjunction with one another and/or with environmental factors. For such polygenic traits, a measure of risk for the polygenic trait, i.e., a polygenic risk score (PRS), may be defined and used to assess the risk for the trait for a given individual. In general, an individual's PRS for a given polygenic disease provides a measure of overall risk of that individual to the disease, with those individuals having a high number of risk loci typically having correspondingly high PRS scores.

A PRS for a respective individual may be generated that represents the genomic profile of the individual based on the known risk loci for a given polygenic trait (e.g., complex disease). By way of example, the alleles associated with risk for the polygenic trait may be identified in the individual's genome (such as via a sequencing or screening process) and used to populate a PRS calculation as determined based on prior genome-wide association studies. In many circumstances, genotyping arrays (i.e., high-pass sequencing) may be employed for calculation of the PRS for a given polygenic trait for an individual. In most instances, the extent of coverage provided by high-pass sequencing may be excessive for what is needed for a PRS calculation. With this in mind, low-pass sequencing may provide an option for calculation of a PRS. Such low-pass sequencing may be performed as a matter of course in various screening applications and may therefore provide a path to providing screening services for an individual via one or more PRS calculations as a secondary function to another screening process for which the low-pass sequencing is performed. However, such low-pass sequencing approaches may, in some instances, introduce confounding factors, such as the presence of DNA that is not that of the individual for which the PRS is being calculated. Such factors may make the use of data derived from such screening processes problematic when used in trying to calculate a PRS for an individual for a given polygenic trait.

### SUMMARY

The presently described techniques provide for the use of low-pass sequencing data in the calculation of a PRS for an individual. As discussed herein, the low-pass sequencing data may be acquired in a context where DNA (e.g., cfDNA) from more than one source is present in the sample and the portion of the DNA attributable to a secondary source may bias the PRS calculation for the primary individual of interest. By way of example, a non-invasive prenatal testing (NIPT) context may involve low-pass sequencing of a sample comprising cfDNA of both the mother and the fetus. In such a context, a PRS calculated based on the low-pass sequencing data for the mother would be biased by the presence of the fetal cfDNA, which would contain paternal DNA. In other contexts the low-pass sequencing data may be derived from an oncological panel or other screening tool in which sequencing data is generated.

With respect to the NIPT example, in this scenario and as described herein the contribution of the father's DNA (via the fetus intermediary) may be removed or reduced so as to remove any bias otherwise affecting the maternal PRS value. In one embodiment, and as discussed in greater detail herein, fetal and maternal DNA fragments (e.g., cfDNA fragments) may be distinguished using suitable techniques, such as fragment length thresholds or other suitable techniques. In certain embodiments, this allows some or all of the fetal sequence data to be excluded from calculation of a PRS specific to the mother. By way of example, in one implementation fragment length may be used to derive a function (e.g., a statistical function, such as a statistical linear function) relating fetal fraction to the respective PRS estimate at each fetal fraction. This function may then be used to the PRS in the absence of a fetal contribution (i.e., at a 0% fetal fraction).

With the preceding in mind, in accordance with certain embodiments disclosed herein, a method is provided for calculating a maternal polygenic risk score. In accordance with this embodiment, a non-invasive prenatal test data set comprising nucleic acid sequence data from a mother and a fetus is accessed or received. The nucleic acid sequence data is filtered using a plurality of minimum fragment length thresholds to generate a respective filtered data set for each minimum fragment length threshold. Each respective filtered data set has a different fetal fraction of contributed nucleic acid sequence data. A polygenic risk score for a polygenic trait of interest is calculated for each respective filtered data set to generate a plurality of polygenic risk scores. A statistical fitting or analysis, such as a linear regression, is performed to determine a relationship (e.g., a linear relationship) between the different fetal fractions and the plurality of polygenic risk scores. The relationship (either linear or non-linear) is extrapolated to a value (e.g., an intercept) corresponding to no contribution of sequence data by the fetus to determine a maternal polygenic risk score. The maternal polygenic risk score is output.

In a further embodiment, a method is provided for calculating a polygenic risk score. In accordance with this embodiment a nucleic acid sequence data set comprising a mixture of sequence data from two sources is accessed or received. The nucleic acid sequence data set is filtered using a plurality of minimum fragment length thresholds to generate a respective filtered data set for each minimum fragment length threshold. Each respective filtered data set has a different proportion of contribution from a first source of the two sources. A polygenic risk score for a polygenic trait of interest is calculated for each respective filtered data set to generate a plurality of polygenic risk scores. A relationship is determined between the different proportions of contribution from the first source and the plurality of polygenic risk scores. Based on the relationship, an unbiased polygenic risk score is determined for a second source of the two sources corresponding to no contribution of sequence data by the first source. The unbiased polygenic risk score is output.

In an additional embodiment, a processor-based system is provided. In accordance with this embodiment, the processor-based system comprises one or more memory structures configured to store data and processor-executable instructions and one or more processors configured to execute the processor-executable instructions. The processor-executable instructions, when executed, cause the one or more processors to performs actions comprising: generating, accessing, or receiving a nucleic acid sequence data set comprising sequence data from a mixture of two sources; filtering the nucleic acid sequence data set using a plurality of minimum fragment length thresholds to generate a respective filtered data set for each minimum fragment length threshold, wherein each respective filtered data set has a different proportion of contribution from a first source of the two sources; calculating a polygenic risk score for a polygenic trait of interest for each respective filtered data set to generate a plurality of polygenic risk scores; determining a relationship between the different proportions of contribution from the first source and the plurality of polygenic risk scores; based on the relationship, determining an unbiased polygenic risk score for a second source of the two sources corresponding to no contribution of sequence data by the first source; and outputting the unbiased polygenic risk score.

The above summary of the present disclosure is not intended to describe each disclosed embodiment or every implementation of the present disclosure. The description that follows more particularly exemplifies illustrative embodiments.

### BRIEF DESCRIPTION OF THE DRAWINGS

These and other features, aspects, and advantages of the present invention will become better understood when the following detailed description is read with reference to the accompanying drawings, in which like characters represent like parts throughout the drawings, wherein:

FIG. 1 depicts a histogram of weights for affected alleles as may be used in a polygenic risk score (PRS) calculation, in accordance with aspects of the present disclosure;

FIG. 2 depicts a conventional process flow for calculating a PRS using high-pass sequencing data;

FIG. 3 illustrates a high-level overview of one example of an image scanning system, in accordance with the present disclosure;

FIG. 4 is a block diagram illustration of an imaging and image processing system, such as for biological samples, in accordance with aspects of the present disclosure;

FIG. 5 is a simplified block diagram of a computer system that can be used to implement aspects of the technology disclosed;

FIG. 6 depicts a process flow for calculating a PRS using low-pass sequencing data, in accordance with the present disclosure;

FIG. 7 depicts an approach for using trio data to generate synthetic sequence data corresponding to a simulated pregnancy, in accordance with the present disclosure;

FIG. 8 graphically depicts PRS score bias as a function of fetal fraction, in accordance with the present disclosure;

FIG. 9 depicts three graphs illustrating the fragment size distribution of fetal and maternal cfDNA in a sample and the effect of fetal fraction on such a distribution, in accordance with the present disclosure;

FIG. 10 illustrates a relationship between fragment size and PRS score, in accordance with the present disclosure;

FIGS. 11A, 111B, and 11C depict, for three samples, plots of minimum fragment length versus fetal fraction, in accordance with the present disclosure;

FIGS. 12A, 12B, and 12C depict, for the three samples of FIG. 11, plots of minimum fragment length versus PRS score, in accordance with the present disclosure;

FIGS. 13A, 13B, and 13C depict, for the three samples of FIG. 11, plots of fetal fraction versus PRS score, in accordance with the present disclosure; and

FIG. 14 depicts a process flow of steps for calculating a maternal PRS with fetal contribution reduced or removed, in accordance with the present disclosure.

### DETAILED DESCRIPTION

The present disclosure relates to the use of low-pass sequencing data to calculate an individual's polygenic risk score (PRS) for a polygenic trait of interest. In particular, the low-pass sequencing data may be acquired based on a screening process unrelated to the polygenic trait of interest. By way of example, low-pass sequencing data acquired as part of non-invasive prenatal testing (NIPT) may be used to calculate one or more PRS scores for the mother (or for the fetus or father) despite the testing data being generated primarily for other purposes. In the case of calculating polygenic risk scores for the mother using NIPT data, in practice the contribution of the father's DNA (via the fetus intermediary) may be removed or reduced, as discussed herein, to improve the value of the PRS in assessing the mother's risk for the polygenic disease or disorder in question. As discussed in greater detail below, removal of sequence data not attributable to the mother may be accomplished using various techniques by which fetal and maternal DNA fragments may be distinguished. By way of example, as discussed herein cfDNA fragment length may be employed in certain embodiments to distinguish likely fetal cfDNA fragments from maternal cfDNA fragments, allowing exclusion of the fetal sequence data from calculation of a PRS specific to the mother. In particular, fragment length may be used, as discussed herein, to derive a function relating fetal fraction to the respective PRS at each fetal fraction and to thereby estimate the PRS in the absence of a fetal contribution (i.e., at a 0% fetal fraction). Though fragment length is described herein as one suitable mechanism for determining a fetal fraction and estimating a corrected PRS, this approach is but one example of suitable approaches for deriving a fetal fraction of a sample and is used to provide a useful, real-world context by which the relevant principles can be described. It should be appreciated, however, that other approaches for calculating a fetal fraction of a sample are available and may be employed to derive a function between fetal fraction and PRS as described herein. By way of example, such other approaches include, but are not limited to use of Y-chromosome fragment data for a male fetus, use of epigenetic (i.e., methylation) patterns, use of allele ratios (as described in greater detail in WO 2012/0142334, which is incorporated by reference herein in its entirety for all purposes), and use of fetal “hot spots” related to portions of the genome that have a higher than expected fetal cfDNA coverage relative to maternal cfDNA (as described in greater detail in U.S. Pat. No. 10,622,094, which is incorporated by reference herein in its entirety for all purposes).

With the preceding in mind, and by way of generalized introduction of certain terminology which may be used herein and/or which may be provide context based on the relevant technical field of endeavor, the following definitions and context are provided. As may be used herein, the term “nucleic acid” is intended to be consistent with its use in the art and includes naturally occurring nucleic acids or functional analogs thereof. Naturally occurring nucleic acids generally have a deoxyribose sugar (e.g., found in deoxyribonucleic acid (DNA)) or a ribose sugar (e.g., found in ribonucleic acid (RNA)). A naturally occurring deoxyribonucleic acid can have one or more bases selected from the group consisting of adenine, thymine, cytosine or guanine and a ribonucleic acid can have one or more bases selected from the group consisting of uracil, adenine, cytosine or guanine.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Example features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation. The term “sensitivity” as used herein is equal to the number of true positives divided by the sum of true positives and false negatives.

The term “specificity” as used herein is equal to the number of true negatives divided by the sum of true negatives and false positives. The term “enrich” herein refers to the process of amplifying nucleic acids contained in a portion of a sample. Enrichment includes specific enrichment that targets specific sequences, e.g., polymorphic sequences, and non-specific enrichment that amplifies the whole genome of the DNA fragments of the sample.

As used herein, the term “each,” when used in reference to a collection of items, is intended to identify an individual item in the collection but does not necessarily refer to every item in the collection unless the context clearly dictates otherwise. As used herein, “providing” in the context of a composition, an article, a nucleic acid, or a nucleus means making the composition, article, nucleic acid, or nucleus, purchasing the composition, article, nucleic acid, or nucleus, or otherwise obtaining, accessing, or acquiring the compound, composition, article, or nucleus. The term “and/or” means one or all of the listed elements or a combination of any two or more of the listed elements. The terms “comprises” and variations thereof do not have a limiting meaning where these terms appear in the description and claims. It is understood that wherever embodiments are described herein with the language “include,” “includes,” or “including,” and the like, otherwise analogous embodiments described in terms of “consisting of” and/or “consisting essentially of” are also provided. Unless otherwise specified, “a,” “an,” “the,” and “at least one” are used interchangeably and mean one or more than one. Also herein, the recitations of numerical ranges by endpoints include all numbers subsumed within that range (e.g., 1 to 5 includes 1, 1.5, 2, 2.75, 3, 3.80, 4, 5, etc.). Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. For any method disclosed herein that includes discrete steps, the steps may be conducted in any feasible order and, as appropriate, any combination of two or more steps may be conducted simultaneously.

With the preceding introductory context in mind, the present disclosure relates to calculating one or more polygenic risk scores for an individual using genome screening data that may be acquired, at least in part, for a separate purpose. By way of introduction to polygenic risk scores, there are instances in which a characteristic or condition (e.g., a disease state) of a person may be monogenic in nature (i.e., attributable to a single mutation at a respective gene or locus). However, in practice many conditions or characteristics of interest are complex and may instead have a multitude of genetic components. Such traits, whether corresponding to a disease state or other non-disease condition, may be referred to as polygenic and may be caused by or associated with hundreds to thousands of genetic variants that act in conjunction with one another and/or with environmental factors. Because it is not a simple matter of analyzing a single locus to evaluate presence, absence, or degree of a polygenic trait, other approaches may be employed as useful tools in assessing such polygenic traits. By way of example, a measure of risk for a polygenic trait, i.e., a polygenic risk score (PRS), which may also be referred to as a polygenic score or genetic risk score, may be defined for a given trait or condition of interest and may be used to assign or define, for a given individual, a genetic risk component for a complex, polygenic disease or trait. Examples of disorders or traits for which the polygenic model may be appropriate, and for which a PRS might be defined and used in making a diagnosis and/or assessing clinical or therapeutic options, include but are not limited to oncological contexts, neurological and/or psychiatric diseases, metabolic diseases (e.g., diabetes), glaucoma, osteoporosis, and so forth.

Every individual will have some number of risk loci for a given polygenic disease and an individual's PRS for that polygenic disease provides a measure of overall risk of that individual to the disease. With this in mind, those individuals having a high number of risk alleles (and/or variants determined to be unusually risky) will typically have correspondingly high polygenic risk scores, which may be used to determine clinical or therapeutic options for the individual if appropriate. In short the polygenic risk score may be considered as corresponding to an individual's likelihood of being affected by a respective polygenic disease now or in the future and/or to the likely severity of the individual's disease state when the disease is present.

Genes associated with a polygenic trait of interest, such as a complex disease, may be identified by genome-wide association studies (GWAS), which are large-scale genetic studies in which samples may be obtained and analyzed from a large number (e.g., hundreds, thousands, tens or hundreds of thousands, and so forth) of individuals. In such studies, not only are a large number of individuals studied, but the sequencing array technology employed allows each individual's genome to be sequenced and analyzed at a large number (e.g., tens of thousands, hundreds of thousands, millions, and so forth) of loci genome-wide. Using such a GWAS approach, the genomes of individuals with the polygenic trait of interest can be compared to those without (e.g., a control group), to determine if the frequency of genetic variants at a locus being reviewed differs between the two groups.

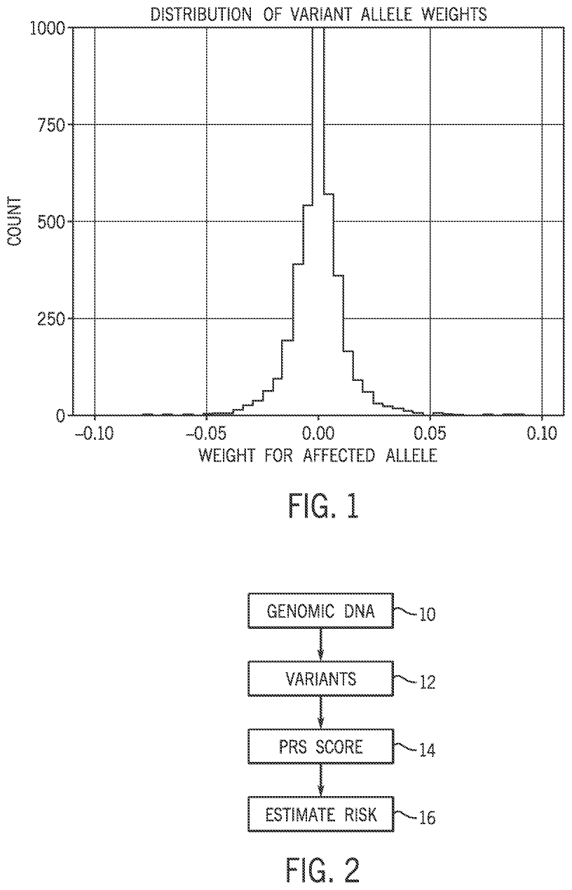

Based on the findings of one or more such GWAS a method of calculating a PRS formula for a polygenic trait of interest (e.g., a PRS definition for that polygenic trait) may be determined. That is, based on GWAS study results, particular variants at identified loci that contribute to the polygenic trait may be identified, along with the varying degrees to which they contribute to the expression (or the degree of expression) of the polygenic trait. Non-linear and/or complex interaction effects associated with various combinations of the variants may also be identified and incorporated into the PRS formula for the polygenic trait of interest. By way of example, Equation 1 represents one such PRS estimation calculation for a given sample (i.e., patient or individual sample) that is based on the number of variants calls identified for the sample (i.e., at loci identified as relevant to the PRS calculation) and the scoring weights for each variant.

$${PRS}_{j} = \frac{{\sum }_{i}^{N} {β}_{i} * {G}_{ij}}{{M}_{j}} \qquad ( 1 )$$

where the PRS calculation is for sample j, there is a weight β for the i^{th} affected allele in the PRS definition, there are a number, G, of affected alleles for variant i in sample j (scaled from 0 to 1), and there are a total (M) of non-missing variants in sample. With respect to the weights β, FIG. 1 graphically illustrates weights for affected alleles (x-axis) versus count (y-axis) as a distribution of allele weights for use in a PRS definition, with negative weights associated with protective single nucleotide polymorphisms (SNPs) and positive weights associated with detrimental SNPs.

With this discussion in mind, a PRS for a respective individual may be generated that incorporates the genomic profile of the individual based on the known risk loci for a given polygenic trait (e.g., complex disease). By way of example, the alleles associated with risk for the polygenic trait may be identified in the individual's genome (such as via a sequencing or screening process) and used estimate the individual's risk based on the PRS definition as determined based on prior genome-wide association studies. This may effectively be, at the simplest end of the spectrum, a simple count of the risk alleles present, or in more complex scoring scenarios may weight the presence or absence of certain alleles based on their assessed contribution to risk and/or may take into account interactions between certain alleles at different loci where the interactions are known to correlate to risk.

While the PRS for an individual with respect to a particular polygenic trait may be used to assess that individual's risk of a disease (or to assess therapeutic or clinical options) as noted above, such scores also have other uses, including, but not limited to, analyzing a population for disease risk and/or selecting samples from a population for studies or focused research efforts. By way of example, PRS values for members of a population may be used to stratify the population according to risk with respect to a complex genetic disease, which may both inform researchers and medical personnel as to the prevalence of a genetic disease or disorder as well as allow effective risk communication strategies to be devised for the population.

In many circumstances, genotyping arrays (i.e., high-pass sequencing) may be employed for calculation of a PRS for a given polygenic trait for an individual. Such high-pass sequencing may be understood to correspond to sequencing of a genome to an average depth of greater than 1× coverage, such as 25× or 30×. A generalized example of one such conventional PRS use case based on high-pass sequencing is shown in FIG. 2. In accordance with this use case, a genomic DNA sample 10 for the individual may be obtained and may undergo high-pass sequencing to obtain the patient's genotype information. Variants 12 that are included in or otherwise relevant to the PRS definition may be identified within the genotype. A PRS score 14 may then be calculated based on the variants 12 identified in conjunction with the PRS definition. The PRS score 14 may be used to estimate risk (step 16) for the individual with respect to the polygenic trait (e.g., polygenic disease or disorder) of interest. Such risk estimation may be further refined to control for demographics and ancestry and may, with respect to relative risk, be based on a comparison to a reference population.

While the approach illustrated in FIG. 2, allows estimation of a PRS score 14, in most instances the high-pass coverage scenario may be excessive for what is needed for a PRS calculation. With this in mind, low-pass sequencing (i.e., sequencing a genome to an average depth equal to or less than 30× coverage, such as coverage levels of 0.25× to 30× (e.g., 0.25×, 0.4×, 0.5×, 0.75×, 1.0×, 5.0×, 10×, 15×, 20×, 25× and so forth)), in combination with or separate from genotype imputation, may provide an option for calculation of a PRS. Such low-pass sequencing may be performed as a secondary benefit or use case with respect to other screening applications and may therefore provide a path to providing screening services for an individual via one or more PRS calculations as a secondary function to another screening process for which the low-pass sequencing is performed.

By way of example, one common screening process that yields low-pass sequencing data is non-invasive prenatal testing (NIPT). NIPT is typically performed using a blood sample drawn from the mother and allows early genetic screening for genetic and chromosomal disorders with no risk to the mother or fetus. NIPT involves analyzing cell-free DNA (cfDNA) from a maternal blood sample, which will comprise a mixture of the fetal and maternal DNA. In practice, this may involve isolating plasma from the maternal blood sample and extracting cfDNA from the plasma for analysis. By way of example, in certain implementations NIPT may be accomplished by performing sequencing (such as via next generation sequencing (NGS) techniques and platforms) to analyze cfDNA fragments derived from the maternal blood sample.

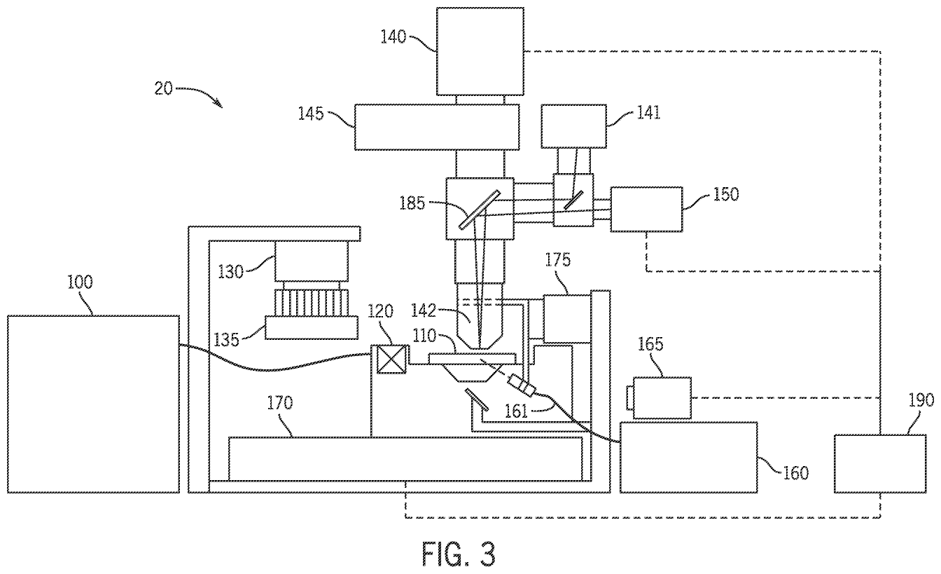

Prior to further discussion of NIPT and of PRS calculation as it pertains to the present techniques, it may be useful to provide a brief, high level overview of an example of suitable systems and functional workflows that may utilize or process samples from which low-pass sequence data may be derived for use in calculating a PRS as described herein. By way of example, FIG. 3 depicts an example of an optical image scanning system 20, such as a NGS system, that may be used to process biological samples, including samples derived from maternal blood for NIPT. With respect to such an imaging system 20, it may be appreciated that such imaging systems typically include a sample stage or support that holds a sample or other object to be imaged (e.g., a flow cell or sequencing cartridge having a patterned surface of spaced apart sample sites) and an optical stage that includes the optics used for the imaging operations.

Turning to FIG. 3, the example image scanning system may include a device for obtaining or producing an image of a region of a flow cell. The example illustrated in FIG. 3 shows an example image scanning system configured in a backlight operational configuration. In the depicted example, subject samples are located on sample container 110, which is positioned on a sample stage 170 under an objective lens 142. Light source 160 and associated optics direct a beam of light, such as laser light, to a chosen sample location on the sample container 110. The sample fluoresces and the resultant light is collected by the objective lens 142 and directed to a photodetector 140 to detect the florescence. Sample stage 170 is moved relative to objective lens 142 to position the next sample location on sample container 110 at the focal point of the objective lens 142.

A fluid delivery module or device 100, as discussed in greater detail below, directs a flow of reagents (e.g., fluorescent nucleotides, buffers, enzymes, cleavage reagents, etc.) to (and through) the sample container 110 and waste valve 120. In some applications, the sample container 110 can be implemented as a flow cell that includes clusters of nucleic acid sequences at a plurality of sample locations on the sample container 110. The samples to be sequenced may be attached to the substrate of the flow cell, along with other optional components. In practice, the plurality of sample locations provided on a surface of the flow cell may be arranged as spaced apart sample sites.

The depicted example image scanning system 20 also comprises temperature station actuator 130 and heater/cooler 135 that can optionally regulate the temperature of conditions of the fluids within the sample container 110. Camera system (e.g., photodetector system 140) can be included to monitor and track the sequencing of sample container 110. The photodetector system 140 can be implemented, for example, as a CCD camera, which can interact with various filters within filter switching assembly 145, objective lens 142. A focusing laser assembly (e.g., focusing laser 150 and focusing detector 141) may also be provided that operates in conjunction with a focus model to provide focus measurements based on the calibration of the focus assembly to a focus model. Light source 160 (e.g., an excitation laser within an assembly optionally comprising multiple lasers) or other light source can be included to illuminate fluorescent sequencing reactions within the samples via illumination through a fiber optic interface 161 (which can optionally comprise one or more re-imaging lenses, a fiber optic mounting, etc.). Low watt lamp 165 and reverse dichroic 185 are also presented in the example shown.

Although illustrated as a backlit device, other examples may include a light from a laser or other light source that is directed through the objective lens 142 onto the samples on sample container 110 (i.e., a front lit configuration). Sample container 110 can be mounted on a sample stage 170 to provide movement and alignment of the sample container 110 relative to the objective lens 142. The sample stage 170 can have one or more actuators to allow it to move in any of three directions. For example, in terms of the Cartesian coordinate system, actuators can be provided to allow the stage to move in the x-, y- and z-directions relative to the objective lens 142. This can allow one or more sample locations on sample container 110 to be positioned in optical alignment with objective lens 142. A focus component 175 is shown in this example as being included to control positioning of the optical components relative to the sample container 110 in the focus direction (typically referred to as the z-axis, or z-direction).

The light emanating from a test sample at a sample location being imaged can be directed to one or more photodetectors 140. Photodetectors can include, for example a CCD camera. An aperture can be included and positioned to allow only light emanating from the focus area to pass to the photodetector(s). The aperture can be included to improve image quality by filtering out components of the light that emanate from areas that are outside of the focus area. Emission filters can be included in filter switching assembly 145, which can be selected to record a determined emission wavelength and to block any stray laser light.

In various examples, sample container 110 (e.g., a flow cell) can include one or more substrates upon which the samples are provided. For example, in the case of a system to analyze a large number of different nucleic acid sequences, sample container 110 can include one or more substrates on which nucleic acids to be sequenced are bound, attached or associated. In various examples, the substrate can include any inert substrate or matrix to which nucleic acids can be attached, such as for example glass surfaces, plastic surfaces, latex, dextran, polystyrene surfaces, polypropylene surfaces, polyacrylamide gels, gold surfaces, and silicon wafers. In some applications, the substrate is within a channel or other area at a plurality of locations formed in a matrix or pattern across the sample container 110.

One or more controllers 190 (e.g., processor or ASIC based controller(s)) can be provided to control the operation of a scanning system, such as the example image scanning system 20 described with reference to FIG. 3. The controller 190 can be implemented to control aspects of system operation such as, for example, scanning, focusing, and imaging operations. In various applications, the controller can be implemented using hardware, software, or a combination of the preceding. For example, in some implementations the controller can include one or more CPUs or processors with associated memory. As another example, the controller can comprise hardware or other circuitry to control the operation. For example, this circuitry can include one or more of the following: field programmable gate arrays (FPGA), application specific integrated circuits (ASIC), programmable logic devices (PLD), complex programmable logic devices (CPLD), a programmable logic array (PLA), programmable array logic (PAL), or other similar processing device or circuitry. As yet another example, the controller can comprise a combination of this circuitry with one or more processors.

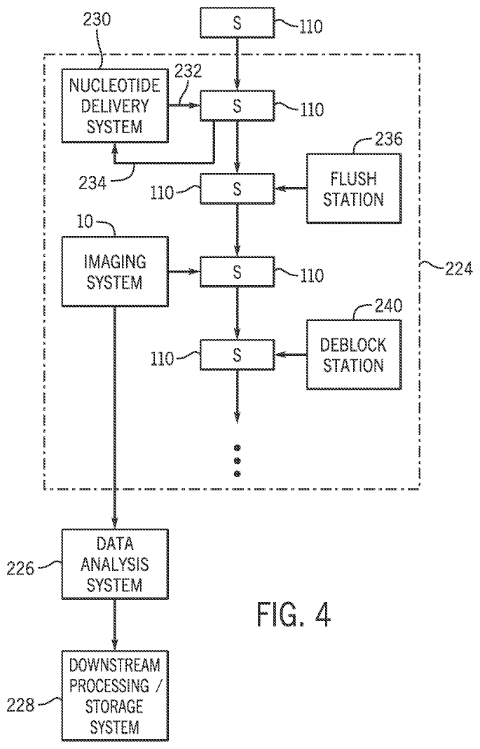

While the preceding description covers components and features of an optical image scanning system 20, such as a sequencing system, FIG. 4 discusses the use of such a system 20 in the context of a functional work flow, such as processing a maternal blood sample for NIPT. This discussion is provided in order to provide useful, real-world context for the subsequent discussion of the generation and use of low-pass sequencing data, such as for calculation of a PRS as discussed herein focus quality metrics. In this manner, it is hoped that the use and significance of low-pass sequence data for PRS calculation as subsequently described will be more fully appreciated.

With this in mind, and turning to FIG. 4, a block diagram illustrating an example work flow in conjunction with system components is provided. In this example, the work flow and corresponding system components may be suitable for processing a maternal blood or plasma sample to derive sequence data. In the illustrated example, molecules (such as nucleotides, oligonucleotides, and other bioactive reagents) may be introduced into a respective sample container 110 that may be prepared in advance. As noted herein, such sample containers 110 may comprise flow cells, sequencing cartridges, or other suitable structures having substrates encompassing sample sites for imaging. The depicted work flow with system components may be utilized for synthesizing biopolymers, such as DNA chains, or for sequencing biopolymers.

Although any of a variety of biopolymers may be processed in accordance with the described techniques, to facilitate and simplify explanation the systems and methods used for processing and imaging in the example context will be described with regard to the processing of nucleic acids. In general, the described work flow will process sample container 110. A single species of biopolymer may be attached to each individual reaction site within the container 110. However, multiple copies of a species of biopolymer can be attached to a reaction site. The pattern, taken as a whole, may include a plurality of different biopolymers attached at a plurality of different sites. Reaction sites can be located at different addressable locations on the same substrate. Alternatively, a patterned surface can include separate substrates each forming a different reaction site. The sites may include fragments of DNA attached at specific, known locations, or may be wells or nanowells in which a target product is to be synthesized. In some applications, the system may be designed for continuously synthesizing or sequencing molecules, such as polymeric molecules based upon common nucleotides.

In the diagrammatical representation of FIG. 4, an analysis system may include a processing system 224 (e.g., a sequencing system or station) designed to process samples provided within sample containers 110, and to generate image data representative of individual sites on the patterned surface. A data analysis system 226 receives the image data and processes the image data in accordance with the present disclosure to extract meaningful values from the imaging data as described herein. A downstream processing/storage system 228, then, may receive this information and store the information, along with imaging data, where desired. The downstream processing/storage system 228 may further analyze the image data or processed data derived from the image data, such as to derive a PRS as discussed herein.

The processing system 224 may employ a biomolecule reagent delivery system (shown as a nucleotide delivery system 230 in the example of FIG. 4) for delivering various reagents to a sample container 110 as processing progresses. The biomolecule reagent delivery system may correspond to the fluid delivery module or device 100 of FIG. 3. Processing system 224 may perform a plurality of operations through which sample container 110 and corresponding samples progress. This progression can be achieved in a number of ways including, for example, physical movement of the sample container 110 to different stations, or loading of the sample container 110 (such as a flow cell) in a system in which the sample container 110 is moved or an optical system is moved, or both, or the delivery of fluids is performed via valve actuation. A system may be designed for cyclic operation in which reactions are promoted with single nucleotides or with oligonucleotides, followed by flushing, imaging and de-blocking in preparation for a subsequent cycle. In a practical system, the sample containers 110 and corresponding samples are disposed in the processing system 224 and an automated or semi-automated sequence of operations is performed for reactions, flushing, imaging, de-blocking, and so forth, in a number of successive cycles before all useful information is extracted from the test sample. Again, it should be noted that the work flow illustrated in FIG. 4 is not limiting, and the present techniques may operate on image data acquired from any suitable system employed for any application. It should be noted that while reference is made in the present disclosure to “imaging” or “image data”, in many practical systems this will entail actual optical imaging and extraction of data from electronic detection circuits (e.g., cameras or imaging electronic circuits or chips), although other detection techniques may also be employed, and the resulting electronic or digital detected data characterizing the molecules of interest should also be considered as “images” or “image data”.

In the example illustrated in FIG. 4, the nucleotide delivery system 230 provides a process stream 232 to the sample containers 110. An effluent stream 234 from the sample containers 110 (e.g., a flow cell) may be recaptured and recirculated, for example, in the nucleotide delivery system 230. In the illustrated example, the patterned surface of the flow cell may be flushed at a flush station 236 (or in many cases by flushing by actuation of appropriate valving, such as waste valve 120 of FIG. 3) to remove additional reagents and to clarify the sample within the sample containers 110 for imaging. The sample containers 110 is then imaged by an imaging system 20 (which may be within the same device). The image data thereby generated may be analyzed, for example, for determination of the sequence of a progressively building nucleotide chain, such as based upon a template.

Following imaging (e.g., at imaging system 20), the sample container 110 may progress to a deblock station 240 for de-blocking, during which a blocking molecule or protecting group is cleaved from the last added nucleotide, along with a marking dye. If the processing system 224 is used for sequencing, by way of example, image data from the imaging system 20 will be stored and forwarded to a data analysis system 226.

The data analysis system 226 may include a general purpose or application-specific programmed computer, which provides a user interface and automated or semi-automated analysis of the image data to determine which of the four common DNA nucleotides may have been last added at each of the sites on a patterned surface. As will be appreciated by those skilled in the art, such analysis may be performed based upon the color of unique tagging dyes for each of the four common DNA nucleotides and, hence, multiple images at one or more light frequencies or combinations of light frequencies may be obtained for each imaged region of the patterned surface.

The data derived from the image data (e.g., sequence and fragment length data) may be further analyzed by a downstream processing/storage system 228, which may store data derived from the image data as described below, as well as the image data itself, where appropriate. By way of example, and as relates to the presently described techniques, the downstream processing/storage system 228 may receive data from the sequencing system that may be utilized to calculate one or more polygenic risk scores for an individual (e.g., a mother who has provided a blood sample for an NIPT). Further, in accordance with the discussion herein, the downstream processing/storage system 228 may execute operations to analyze the sequence data (such as based on fragment length or other criteria) so as to characterize fragments as being attributable to the mother or fetus and may, based upon such characterization, correct for the presumptive fetal contribution from the PRS calculations. One or more of the operations of analysis of the sequence data, characterization of fragments as corresponding to fetal cfDNA or maternal cfDNA, and/or calculation of a maternal (or paternal) PRS based upon these characterizations may be implemented on one or more downstream processing/storage systems 228 as described herein, such as by execution of stored routines on the components of such a system based upon sample specific patient data (e.g., sequence/variant data 250 and/or fragment length data 254).

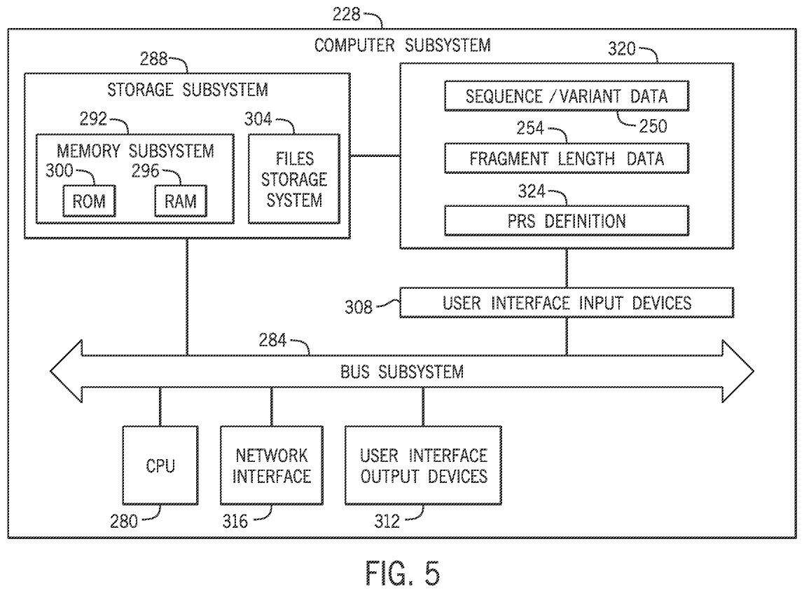

With this in mind, an example of one such possible downstream processing/storage system 228 is provided in FIG. 5. In this example system, a high-level hardware architecture is described for reference. Such hardware may be physically embodied as one or more computer systems (e.g., servers, workstations, and so forth). Examples of components which may be found in such a processing/storage system 228 are illustrated in FIG. 5, though it should be appreciated that the present example may include components not found in all embodiments of such a system or may not illustrate all components that may be found in such a system. Further, in practice aspects of the present approach may be implemented in part or entirely in a virtual server environment or as part of a cloud platform. However, in such contexts the various virtual server instantiations will still be implemented on a hardware platform as described with respect to FIG. 5, though certain functional aspects described may be implemented at the level of the virtual server instance.

With this in mind FIG. 5 is a simplified block diagram of a computer system that can be used to implement the technology disclosed. Such a computer system typically includes at least one processor (e.g., CPU) 280 that communicates with a number of peripheral devices via bus subsystem 284. These peripheral devices can include a storage subsystem 288 including, for example, memory devices 292 (e.g., RAM 296 and ROM 300) and a file storage subsystem 304, user interface input devices 308, user interface output devices 312, and a network interface subsystem 316. The input and output devices allow user interaction with computer system (e.g., processing/storage system 228). Network interface subsystem 316 provides an interface to outside networks, including an interface to corresponding interface devices in other computer systems.

In one implementation in which a computer system such as shown in FIG. 5 is used to calculate a PRS based on low-pass sequencing data, including NIPT data having a both maternal and fetal cfDNA contributions, interface and user options allowing selection and/or manipulation of the relevant data sets and formulas 320 (e.g., sequence and variant data 250 derived from a respective sample, fragment length data 254, and/or a PRS definition (e.g., formula) 324 to be utilized in calculating a PRS) may be provided. As shown such data and calculation operations may be received and or stored so as to be communicably linked to the storage subsystem 288 and user interface input devices 308.

In the context of the depicted computer system, the user interface input devices 308 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” may be construed as encompassing all possible types of devices and ways to input information into computer system.

User interface output devices 312 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” may be construed as encompassing all possible types of devices and ways to output information from computer system to the user or to another machine or computer system.

Storage subsystem 288 stores programming and data constructs that provide the functionality of some or all of the modules and methods described herein. These software modules are generally executed by a processor 280 alone or in combination with other processors 280.

Memory 292 used in the storage subsystem 288 can include a number of memories including a main random-access memory (RAM) 296 for storage of instructions and data during program execution and a read only memory (ROM) 300 in which fixed instructions are stored. A file storage subsystem 304 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 304 in the storage subsystem 288, or in other machines accessible by the processor 280.

Bus subsystem 284 provides a mechanism for letting the various components and subsystems of computer system communicate with each other as intended. Although bus subsystem 284 is shown schematically as a single bus, alternative implementations of the bus subsystem 284 can use multiple busses.

The computer system itself can be of varying types including a personal computer, a portable computer, a workstation, a computer terminal, a network computer, a television, a mainframe, a stand-alone server, a server farm, a widely-distributed set of loosely networked computers, or any other data processing system or user device. Due to the ever-changing nature of computers and networks, the description of computer system depicted in FIG. 5 is intended only as an example for purposes of illustrating the functionality and types of components associated with the technology disclosed. Many other configurations of computer system are possible having more or less components or different components than the computer system depicted in FIG. 5.

With the preceding in mind, and as noted above, aspects of the approaches described herein contemplate the use of low-pass sequence data to calculate a PRS for an individual. By way of a real-world example, such low-pass sequence data may be obtained for a mother as part of a non-invasive prenatal test (NIPT). It should be appreciated, however, that while NIPT is presented as one use case scenario, other low-pass sequencing derived data, such as may be generated by various diagnostic specific screening operations or tests such as oncological panels, may also be employed to calculate a PRS for a given individual. However, for the purpose of illustration, and to further describe techniques for removing or limiting the effects of DNA from another individual, examples related to the use of NIPT low-pass sequence data for calculating a PRS score will be primarily described.

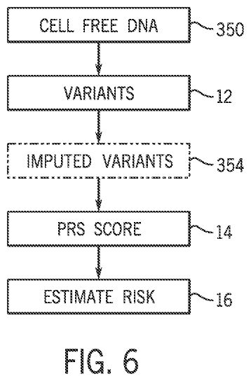

With this in mind, and turning to FIG. 6, an example of a NIPT-based use case for calculating a PRS score is illustrated. In this example, cfDNA 350 is acquired from a maternal blood sample and low-pass sequenced (e.g., sequence coverage of approximately 0.25×). Variants 12 implicated by the respective PRS definition are identified. In the depicted example, a genotype imputation step, may also be performed to generate a set of imputed variants 354. As used herein, such a genotype imputation step may be appropriate in the context of low-pass sequencing in order to predict or impute genotypes that, due to low coverage, are not directly assayed in the sample or are assayed but at an insufficient depth for reliable variant calling. In particular, such imputation algorithms may compare single nucleotide polymorphisms (SNPs) or other identified sequences with reference whole genome sequences to identify matching or missing segments that are missing from the data due to the low coverage of the low-pass sequencing. In context, based on the observed sequence data and variants, other variants, i.e., imputed variants 354, may be statistically assumed to be present due to observed associations within a larger population of whole genome data. In this manner, missing or low-quality variant calls within a low coverage dataset may be filled in or replaced based on known or observed relationships within the whole genome data observed for a population.

The variants 120 and imputed variants 354 may be used to calculate a PRS score 14 based on the previously determined PRS definition. The PRS score 14 may be used to estimate risk (step 16) for the individual for the polygenic trait (e.g., polygenic disease or disorder). Such risk estimation may be further refined to control for demographics and ancestry and may, with respect to relative risk, be based on a comparison to a reference population.

As may be appreciated, the approach outlined in FIG. 6, when employed in conjunction with NIPT data, presents a challenge that may be absent in conventional approaches employing high-pass sequence data to estimate a PRS, namely the fetal cfDNA contribution that will be present in an NIPT sample. As discussed below, this challenge may be addressed to make such use of low-pass sequence and NIPT data useful for PRS estimation for one or both parents.

With respect to the fetal fraction contribution that may be introduced by the use of NIPT data to calculate a maternal PRS, it may be appreciated that the greater the fetal fraction of cfDNA in the tested sample, the greater the chance that a fetal haplotype will be observed, thereby biasing the PRS score higher or lower than what would be observed absent the fetal contribution. This prospect was tested using a synthetic data set generated using 32 known genome trios (i.e., a mother, father, child). In particular, synthetic data was generated in the form of simulated pregnancies derived using the trio data.

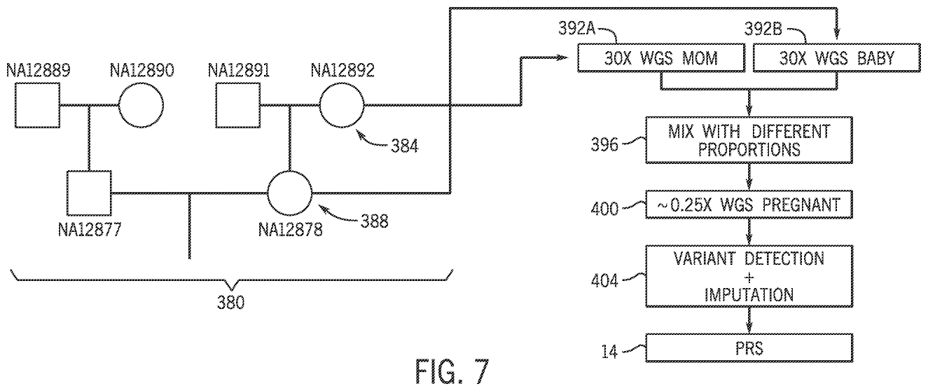

An example of this approach for synthesizing simulated pregnancy data is depicted in FIG. 7. In this example trio relationships 380 for mother, father and child are shown on the left. For a given mother 384 and child 388, whole genome sequence data (392A, 392B) acquired via high-pass sequencing (e.g., 30× coverage) is available. Using the whole genome sequence data for a given mother 384 and child 388, a synthetic whole genome sequence data set may be generated having a different proportion of maternal to fetal sequence data (e.g., 0%, 5%, 10%, 15%, and so on). Conceptually, such a mixed synthetic data set may be equated to the whole genome sequence data present within a maternal blood sample.

The synthetic mixed contribution data set may be analytically processed in silico (e.g., sampled) in the equivalent of a low-pass sequencing (e.g., 0.25× coverage) operation (step 400) corresponding to the low-pass coverage obtained in an NIPT context. As may be appreciated, this may correspond to performing an NIPT of the simulated pregnancy. In accordance with the present approach, the results of this low-pass sequencing step may be processed (step 404) to identify variants of interest and/or to perform genotype imputation, as discussed herein. Based on the identified and imputed variants from the synthetic data set, a PRS score 14 may be calculated based on a known PRS definition. As may be appreciated, the above approach may be done for varying fetal fractions (e.g., 0%, 5%, 10%, 15% fetal fraction) to observe the effect of fetal fraction on PRS using synthesized data. As may also be appreciated, in the described context, the fetal fraction continues to bias (upward or downward) the PRS 14 relative to what would be observed using the sequence data 392A for the mother 384 alone. That is, the presence of fetal DNA, in both synthetic and non-synthetic data contexts, causes the maternal PRS to trend toward the fetal PRS. The PRS bias will depend on the amount of fetal DNA included as part of the sample in the synthetic data context (which serves as a surrogate for fetal fraction) and on the magnitude and direction of difference between the fetal PRS and the maternal PRS.

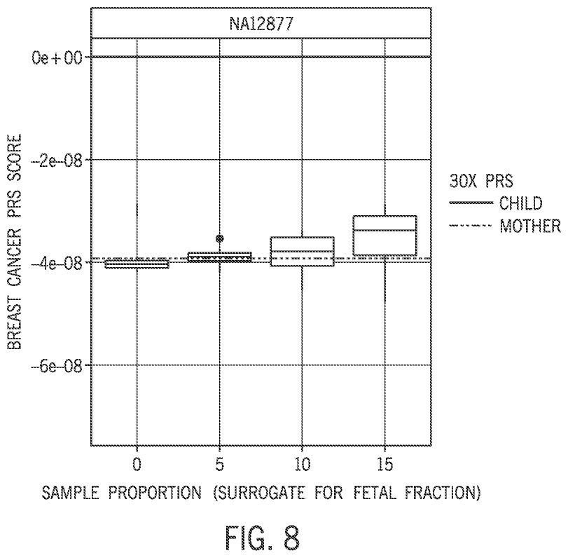

This concept is illustrated in FIG. 8, in which breast cancer PRS scores for a simulated pregnancy are illustrated as a plot where fetal DNA sample proportions (i.e., fetal fraction) of 0%, 5%, 10%, and 15% are plotted on the X-axis and PRS score box plots are plotted along the y-axis. The fetal PRS determined using the fetal WGS 392B is shown as a horizontal line at 0×10⁰ while the maternal PRS determined using the maternal WGS 392A is shown as a horizontal line at −4×10⁻⁸. As shown in FIG. 8, as the sample proportion of fetal DNA (i.e., the fetal fraction) increases along the x-axis, the PRS score for the mixed sample is pulled away from the maternal PRS and toward the fetal PRS. As also shown in the example of FIG. 8, the spread of the observed values represented by the box plots also increases as fetal contribution increases.

This fetal contribution effect may therefore introduce some level of bias and uncertainty in maternal PRS values estimated from NIPT data. To address this bias and uncertainty, the presently disclosed techniques may be used to provide a correction to fetal fraction bias, allowing a single individual's PRS score to be more accurately estimated from NIPT derived data or, as may be appreciated, other low-pass sequencing data derived from mixed contribution sources (e.g., donor-donee contexts and so forth). Further, while the preceding discussion and following examples and explanation primarily describe estimation of a maternal PRS from an NIPT data set by correcting for the fetal contribution, in practice the same techniques may instead be used to estimate a PRS for the paternal contribution and/or for the fetus as the relative contributions each genotype are parseable as described herein, allowing the contributions of others to be identified and removed. That is, estimation of the maternal contribution and correction for the maternal contribution may similarly allow a paternal PRS to be estimated.

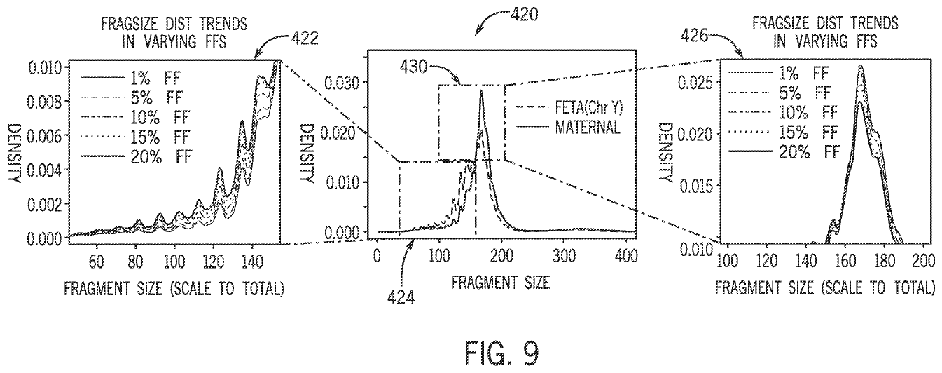

With the preceding in mind, certain implementations of the presently disclosed techniques utilize fragment size as a way of differentiating fragments contributed by the fetus relative to those contributed by the mother, such as in a blood sample processed for NIPT. Turning to FIG. 9, the basis for this approach is illustrated. In particular, FIG. 9 illustrates three graphs based on read size filtering of an NIPT sample. As shown in these figures, fetal cfDNA fragments are typically smaller than maternal cfDNA fragments. Based on this observation, fetal contribution may be reduced by filtering out reads below a threshold size.

Turning to FIG. 9, the central graph 420 illustrates a plot of fetal cfDNA fragments (determined as being Y chromosome fragments) and maternal fragments. In graph 420, cfDNA fragment size (measured in base pairs (BP)) is on the x-axis while density is illustrated on the y-axis. As shown in graph 420, in the depicted sample a transition can be observed at approximately 160 bp at which point the sample transitions from being predominantly fetal fragments to being predominantly maternal fragments.

Aspects of this trend are further illustrated with respect to graph 422, which depicts a breakdown based on fetal fraction of the area enclosed by outline 424 on graph 420 and graph 426, which depicts a breakdown based on fetal fraction of the area enclosed by outline 430 on graph 420. In the two breakdown graphs 422 and 426, plots are illustrated for fetal fractions of 1%, 5%, 10%, 15%, and 20% along an x-axis corresponding to fragment size and a y-axis corresponding to the probability density of observing the fragment size. Graph 422 covers a range of fragment sizes (as illustrated on the x-axis) from approximately 40 to 160, representing the region at which fetal fragments are at greater density than maternal fragments in graph 420, i.e., prior to the transition. In graph 422 it can be observed that the higher the fetal fraction the greater the probability density at the respective fragment sizes. This is in contrast to what may be observed in graph 426 illustrating the same fetal fraction values but after the transition point observed at approximately 160 bp in graph 420. As shown in graph 426 at approximately 160 bp and beyond it can be observed that the lower the fetal fraction the greater the probability density at respective fragment sizes. Taken together it may be understood that fetal cfDNA fragments are typically smaller in size than maternal cfDNA fragments and that fetal fraction is a factor in determining the proportion of fetal cfDNA fragments at a given fragment size. For the represented sample, the transition of approximately 160 bp corresponds to the threshold below which the sample is enriched for fetal cfDNA fragments and above which maternal cfDNA fragments predominate.

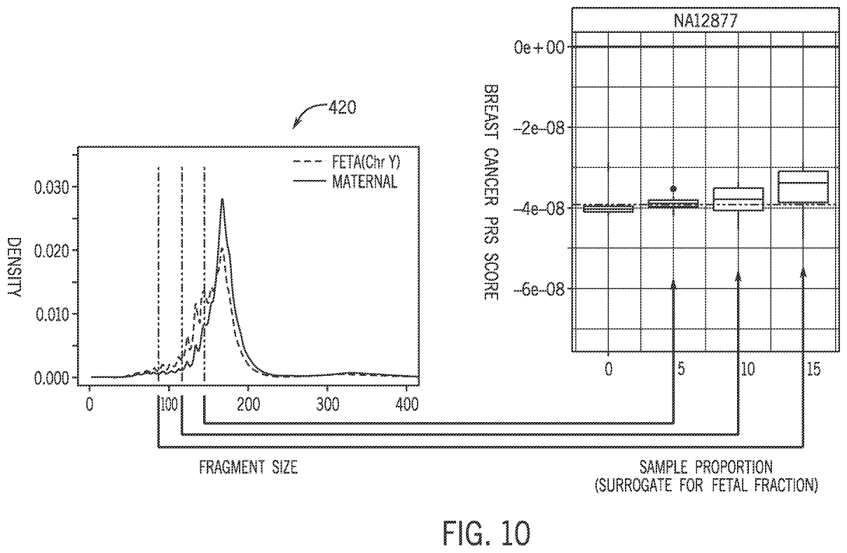

This observation may be utilized in the present context to derive a correction for fetal fraction bias to allow a PRS to be estimated for a mother using low-pass sequencing data derived from NIPT data that also includes fetal cfDNA as part of the sample. In particular, for a given sample, multiple fragment length thresholds may be determined that correspond to a series of fetal fractions in order to generate a fetal fraction titration series that may then be used to derive a trend line, as discussed below. Aspects of this are conceptually illustrated in FIG. 10, which leverages the graphs and plots of FIGS. 8 and 9 to illustrate the relationship between fragment size and fetal fraction. The trend determined from the titration series can subsequently be used to correct for PRS estimation bias attributable to the fetal fraction.

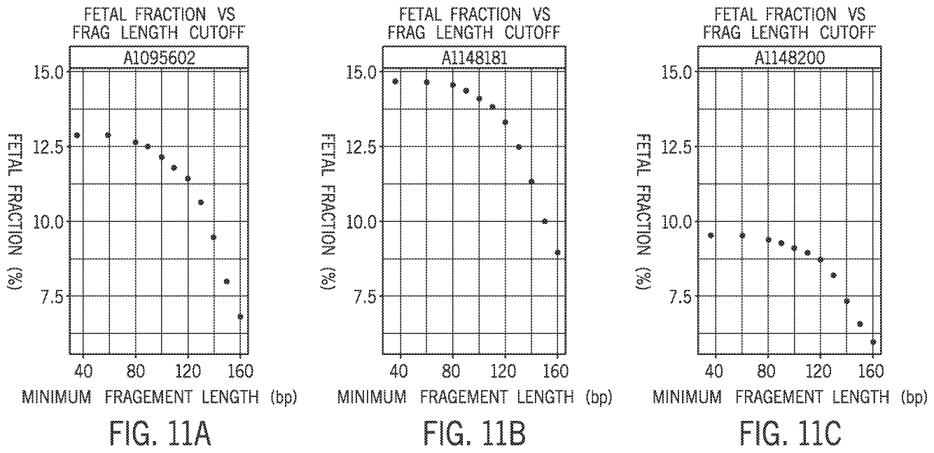

Examples of such titration series for three separate samples are shown in FIGS. 11A, 11B, and 11C. In the depicted graphs points are plotted corresponding to fetal fraction (y-axis) at different minimum fragment lengths (x-axis). In particular, in this example the x-axis shows the minimum fragment length threshold used prior to processing the sample through the NIPT pipeline. The y-axis shows the fetal fraction estimate derived from chromosomes X and Y in male fetuses. As illustrated in this example, as the minimum fragment size used to process the sample increases, the fetal fraction decreases. This occurs because the fetal cfDNA is removed at a faster rate than maternal cfDNA when shorter fragments are omitted.

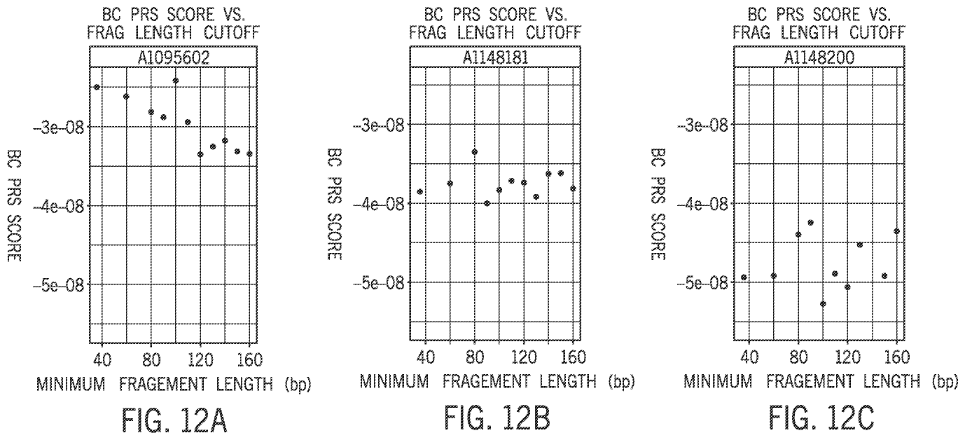

Turning to FIGS. 12A, 12B, and 12C, the trend in calculated PRS as fetal fraction is decreased (by removing fetal cfDNA from consideration based upon size threshold criteria) can also be plotted based on this titration series, as shown for the same three samples. In the depicted graphs points are plotted corresponding to breast cancer PRS calculations in conjunction with different minimum fragment lengths (x-axis).

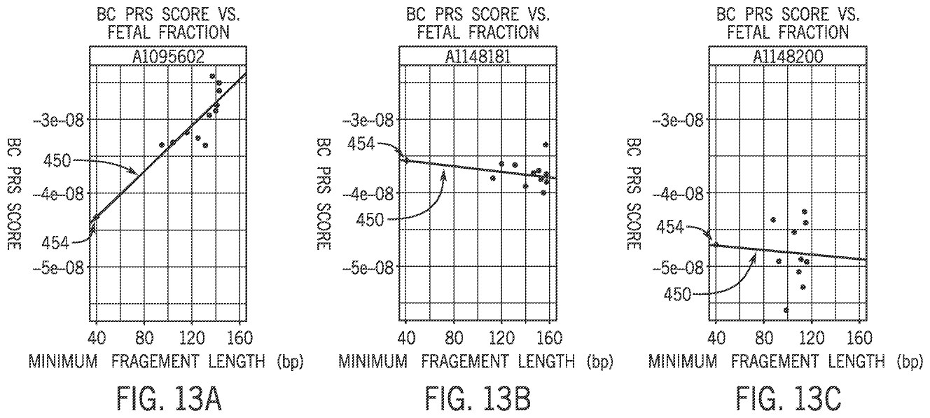

The relationship between breast cancer PRS values in this example and the fetal fraction titration created by filtering the minimum fragment length of respective single samples is illustrated in FIGS. 13A, 13B, and 13C. As shown in these figures a linear trend (such as may be determined using linear regression) between PRS value and the fetal fraction can be used to extrapolate to a PRS value corresponding to 0% fetal fraction. This would be an estimate of the maternal breast cancer risk in the absence of fetal cfDNA in the NIPT sample. That is, a trend line 450 may be fit relating PRS value (here for breast cancer) (y-axis) to fetal fraction (x-axis). In this example, the y-intercept 454 of the trend line 450 corresponds to the PRS value at 0% fetal contribution. In this manner the trend line 450 may be used to estimate the maternal PRS for the polygenic trait or disease of interest, which will correspond to the y-intercept 454 of the trend line 450. It may be noted that this technique is not suitable for use with genomic DNA, such as the synthetic pregnancy data described herein, due to the genomic DNA not having the same fragment length properties as cfDNA as described in the present example. Due to the difference in fragment length properties between genomic DNA and cfDNA, the genomic DNA cannot be filtered to change the fetal fraction as in the present example.

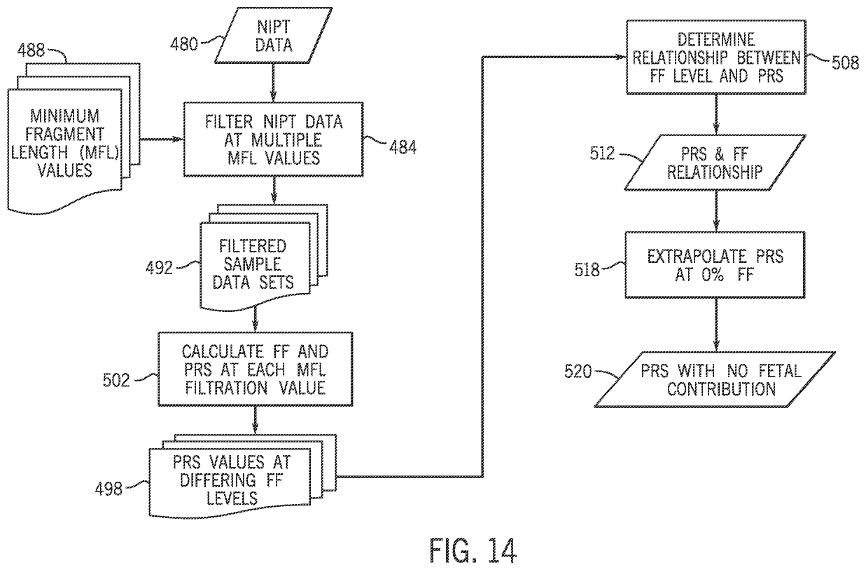

The preceding provides a visual walkthrough of certain aspects of the present approach to help facilitate explanation. In terms of a practical work flow of how one implementation of the steps may be employed, FIG. 14 depicts steps of a workflow for calculating a maternal PRS from an NIPT sample with the fetal contribution to the PRS reduced or removed. In the depicted example, NITP data 480 is generated or otherwise accessed, such as subsequent to an NIPT screening. The NIPT data 480 is filtered (step 484) based on multiple minimum fragment length (MFL) values 488 (e.g., 25 bp, 30 bp, 35 bp . . . 150 bp, 155 bp, 160 bp, and so forth) so as to generate a corresponding filtered sample data set 492 for each MFL value in which the respective data set 492 has had fragments below the respective MFL value (i.e., threshold) removed.

A PRS 498 is calculated (step 502) for each respective filtered sample data set 492 such that a PRS 498 is generated for each fetal fraction level of interest. A relationship 512 (e.g., a linear relationship) is then determined (step 508) between fetal fraction level and PRS. By way of example, in one embodiment a linear regression may be performed at step 508 to determine the relationship 512. Based on the determined relationship between PRS and fetal fraction, a PRS 520 with no fetal contribution can be determined, such as by extrapolating (step 518) a linear relationship between PRS and fetal fraction to derive a PRS value at zero fetal contribution.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for calculating a polygenic risk score, comprising:
accessing or receiving a nucleic acid sequence data set comprising a mixture of sequence data from two sources;
filtering the nucleic acid sequence data set using a plurality of minimum fragment length thresholds to generate a respective filtered data set for each minimum fragment length threshold, wherein each respective filtered data set has a different proportion of contribution from a first source of the two sources;
calculating a polygenic risk score for a polygenic trait of interest for each respective filtered data set to generate a plurality of polygenic risk scores;
determining a relationship between the different proportions of contribution from the first source and the plurality of polygenic risk scores;
based on the relationship, determining an unbiased polygenic risk score for a second source of the two sources corresponding to no contribution of sequence data by the first source; and
outputting the unbiased polygenic risk score.

2. The method of claim 1, wherein the nucleic acid sequence data set comprises a low-pass sequencing data set.

3. The method of claim 1, wherein the nucleic acid sequence data set comprises a non-invasive prenatal test (NIPT) sequence data set.

4. The method of claim 1, wherein the nucleic acid sequence data set comprises variants and imputed variants.

5. The method of claim 1, wherein the distribution of fragment lengths for each of the two sources differs.

6. The method of claim 1, wherein the relationship is a linear relationship.

7. The method of claim 1, wherein determining the relationship comprises performing a statistical fitting or analysis.

8. The method of claim 1, wherein determining the unbiased polygenic risk score comprises extrapolating a statistical fitting describing the relationship to a value that corresponds to no contribution of sequence data by the first source.

9. A processor-based system, comprising:
one or more memory structures configured to store data and processor-executable instructions; and
one or more processors configured to execute the processor-executable instructions, wherein the processor-executable instructions, when executed, cause the one or more processors to performs actions comprising:
generating, accessing, or receiving a nucleic acid sequence data set comprising sequence data from a mixture of two sources;
filtering the nucleic acid sequence data set using a plurality of minimum fragment length thresholds to generate a respective filtered data set for each minimum fragment length threshold, wherein each respective filtered data set has a different proportion of contribution from a first source of the two sources;
calculating a polygenic risk score for a polygenic trait of interest for each respective filtered data set to generate a plurality of polygenic risk scores;
determining a relationship between the different proportions of contribution from the first source and the plurality of polygenic risk scores;
based on the relationship, determining an unbiased polygenic risk score for a second source of the two sources corresponding to no contribution of sequence data by the first source; and
outputting the unbiased polygenic risk score.

10. The processor-based system of claim 9, wherein the nucleic acid sequence data set comprises a low-pass sequencing data set.

11. The processor-based system of claim 9, wherein the nucleic acid sequence data set comprises a non-invasive prenatal test (NIPT) sequence data set.

12. The processor-based system of claim 9, wherein the distribution of fragment lengths for each of the two sources differs.

13. The processor-based system of claim 9, wherein determining the unbiased polygenic risk score comprises extrapolating a statistical fitting describing the relationship to a value that corresponds to no contribution of sequence data by the first source

14. A method for calculating a maternal polygenic risk score, comprising:
accessing or receiving a non-invasive prenatal test data set comprising nucleic acid sequence data from a mother and a fetus;
filtering the nucleic acid sequence data using a plurality of minimum fragment length thresholds to generate a respective filtered data set for each minimum fragment length threshold, wherein each respective filtered data set has a different fetal fraction of contributed nucleic acid sequence data;
calculating a polygenic risk score for a polygenic trait of interest for each respective filtered data set to generate a plurality of polygenic risk scores;
performing a linear regression to determine a linear relationship between the different fetal fractions and the plurality of polygenic risk scores;
extrapolating the linear relationship to an intercept corresponding to no contribution of sequence data by the fetus to determine a maternal polygenic risk score; and
outputting the maternal polygenic risk score.

15. The method of claim 14, wherein the nucleic acid sequence data comprises low-pass sequencing data.

16. The method of claim 14, wherein the polygenic trait of interest comprises a disease or disorder.

17. The method of claim 14, wherein the nucleic acid sequence data comprises observed variants and imputed variants.

18. The method of claim 14, wherein below a transition fragment length the proportion of fetal fragments exceed the proportion of maternal fragments.

19. The method of claim 14, wherein the nucleic acid sequence data is derived from cell-free DNA (cfDNA) fragments.

20. The method of claim 19, wherein the minimum fragment length thresholds filter out data from cfDNA fragments below the respective minimum fragment length thresholds.
